(19) United States
(12) Patent Application Publication
(10) Pub. No.: US 2009/0137949 A1
(43) Pub. Date: May 28, 2009

(54) NEEDLE-FREE INJECTION DEVICE WITH NOZZLE AUTO-DISABLE

(75) Inventors: Sergio Landau, Laguna Niguel, CA (US); Daniel E Williamson, Sherwood, OR (US); John R. Marshall, Beaverton, OR (US)

Correspondence Address: KOLISCH HARTWELL, P.C., 200 PACIFIC BUILDING, 520 SW YAMHILL STREET, PORTLAND, OR, 97204, US

(73) Assignee: Bioject Inc., Tualatin, OR (US)

(21) Appl. No.: 11/945,205

(22) Filed: Nov. 26, 2007

(51) Int. Cl. A61M 5/30

(52) U.S. Cl. 604/70

(57) ABSTRACT

A nozzle assembly for a needle-free injection device. The nozzle assembly includes a nozzle body including an injectate chamber and one or more outlet orifices and a plunger configured to move through the injectate chamber toward the one or more outlet orifices. In some embodiments, the plunger includes a first portion and a second portion removably joined by a frangible region. In some embodiments, the plunger includes extensions configured to couple the plunger to a drive assembly of a needle-free injection device.

### CROSS-REFERENCE TO RELATED APPLICATIONS

This application relates to U.S. patent application entitled “NEEDLE-FREE INJECTION DEVICE WITH AUTO-DISABLE,” filed Nov. 26, 2007, the disclosure of which is incorporated herein by reference.

### BACKGROUND

Needle-free injection systems provide an alternative to standard fluid delivery systems, which generally use a needle adapted to penetrate the outer surface of a target. Typically, needle-free injection systems are designed to eject the fluid from a fluid chamber with sufficient pressure to allow the fluid to penetrate the target to the desired degree. For example, common applications for needle-free injection systems include delivering intradermal, subcutaneous and intramuscular injections into or through a recipient's skin. For each of these applications, the fluid must be ejected from the system with sufficient pressure to allow the fluid to penetrate the tough exterior dermal layers of the recipient's skin.

Examples of needle-free injection systems and components are found in U.S. Pat. Nos. 4,592,742, 4,596,556, 4,790,824, 4,940,460, 4,941,880, 5,062,830, 5,064,413, 5,312,335, 5,312,577, 5,383,851, 5,399,163, 5,503,627, 5,505,697, 5,520,639, 5,746,714, 5,782,802, 5,893,397, 5,993,412, 6,096,002, 6,132,395, 6,216,493, 6,264,629, 6,319,224, 6,383,168, 6,415,631, 6,471,669, 6,506,177, 6,572,581, 6,585,685, 6,607,510, 6,641,554, 6,645,170, 6,648,850, 6,623,446, 6,676,630, 6,689,093 6,709,427, 6,716,190, 6,752,780, 6,752,781, 6,783,509, 6,935,384, 6,942,645, 6,979,310, 6,981,961, 7,056,300 and 7,156,823; U.S. Patent Application Publication Nos. 2005/0119608 and 2006/0189927; and International Publication No. WO 00/72908, the disclosures of which are incorporated herein by reference, in their entirety and for all purposes.

### SUMMARY

The present disclosure is directed to nozzle assemblies for needle-free injection devices. The disclosed nozzle assembly includes a nozzle body including an injectate chamber and one or more outlet orifices and a plunger configured to move through the injectate chamber toward the one or more outlet orifices. In some embodiments, the plunger includes a first portion and a second portion removably joined by a frangible region. In some embodiments, the plunger includes deformable extensions configured to selectively couple the plunger to a drive assembly of a needle-free injection device.

The advantages of the disclosed nozzle assembly may be understood more readily after a consideration of the drawings and the Detailed Description.

### BRIEF DESCRIPTION OF THE DRAWINGS

FIG. 1 is a cross-sectional view of an example of a nozzle assembly coupled with an example of a needle-free injection device having a delivery system and an actuation system.

FIG. 2 illustrates a nozzle assembly being coupled to a delivery system of a needle-free injection device, the nozzle assembly includes a nozzle body and a plunger.

FIG. 3 illustrates the nozzle assembly of FIG. 2 being retracted by the delivery system to draw a dose of injectate into the nozzle assembly.

FIG. 4 illustrates the nozzle assembly of FIG. 3 after delivery of an injection in which the plunger breaks along a frangible region such that a portion of the plunger remains in the nozzle body.

FIG. 5 illustrates an example of a frangible region for a plunger.

FIG. 6 illustrates an example of a nozzle assembly including an intradermal nozzle assembly and a vial adapter.

FIG. 7 illustrates a cross-sectional view of an intradermal nozzle assembly.

FIG. 8 illustrates a nozzle assembly including a plunger having extensions to couple the plunger to a ram of a delivery system; the ram includes a curved portion.

FIG. 9 illustrates a nozzle assembly including a plunger having extensions to couple the plunger to a ram of a delivery system; the ram includes a cutting portion.

FIG. 10 illustrates the nozzle assembly of FIG. 9 with the extensions deformed away from the ram.

FIG. 11 illustrates a nozzle assembly including a plunger having extensions to couple the plunger to a ram of the delivery system; the ram includes an angled portion.

FIG. 12 illustrates the nozzle assembly of FIG. 11 with the extensions deformed away from the ram.

### DETAILED DESCRIPTION

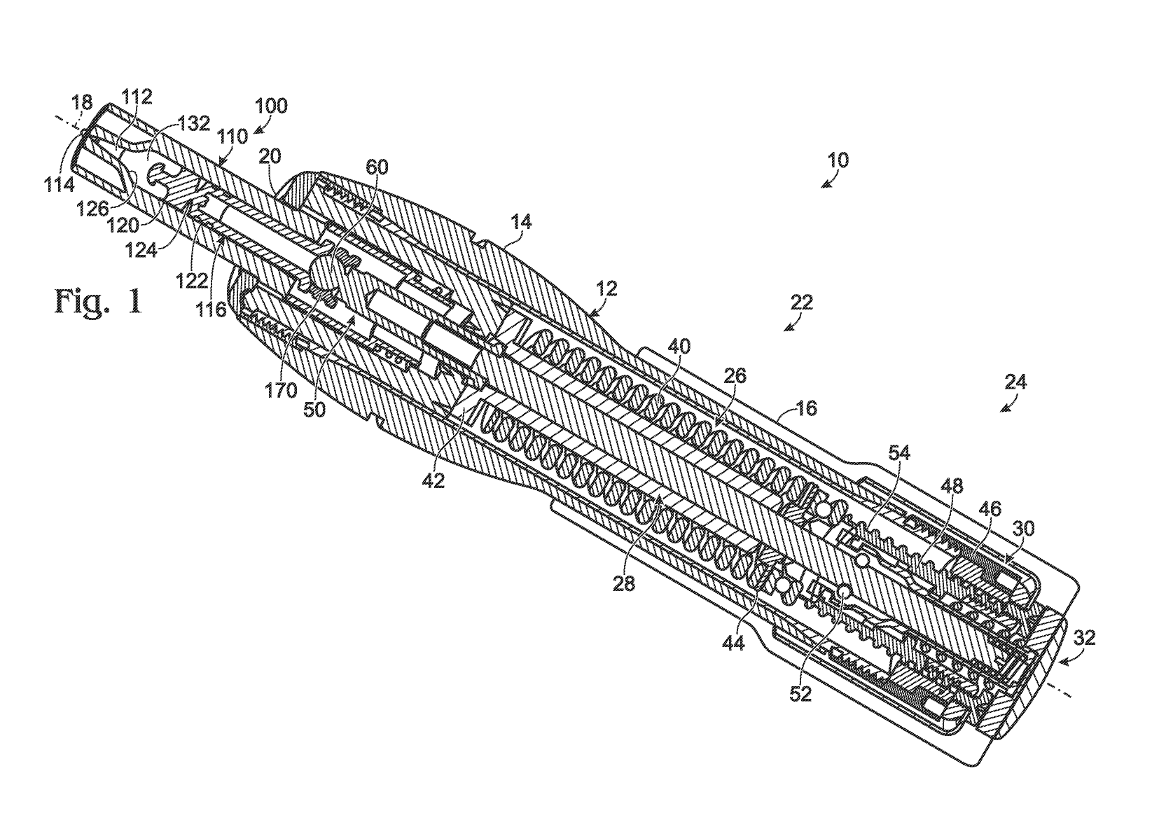

FIG. 1 illustrates an example of a needle-free injection device 10 and a nozzle assembly 100. Although the disclosed injection device is intended to be reusable, the nozzle assembly includes various auto-disable features to restrict reuse of the nozzle assembly. The nozzle may be replaced, for example, after every injection or after a set number of injections.

Device 10 includes a body 12 to enclose various systems used to effect an injection. The body is typically sized and shaped to be comfortably held in a user's hand and may take any suitable configuration. Body 12 may be formed from injection-molded plastic, though various other materials and fabrication methods may be suitable.

As illustrated in FIG. 1, body 12 may be comprised of various subsections, such as housings 14, 16. The housings may be configured to move relative to one another to actuate the various systems. In the example shown in FIG. 1, one or more of the housings may be rotatable relative to another housing and/or rotatable about a central axis 18 to actuate various assemblies of the device.

The body includes an opening 20 in an end of the device to receive the nozzle assembly. The body may include other apertures, such as one or more view ports, to provide feedback or instructions to a user of the device. The apertures may align with indicia, such as arrows or text, which instruct a user in proper operation of the device or convey information to a user, such as the current configuration or status of the device.

Nozzle assembly 100 is configured to be selectively coupled to the delivery system. The nozzle assembly houses an injectate and provides an interface with a recipient's skin. As illustrated in FIGS. 1-4, nozzle assembly 100 includes a nozzle body 110 forming an injectate chamber 112 with one or more outlet orifices 114. The nozzle assembly further includes a plunger 116 configured to move through the injectate chamber toward the orifice to expel an injectate.

Device 10 may include one or more systems to effect an injection. For example, the device of FIG. 1 includes a delivery system 22 and an actuation system 24. Delivery system 22 provides an interface for delivery of an injectate to a recipient and delivers an injection by expelling the injectate from the device. Delivery system 22 is configured to expel a volume of fluid from the device, such as a drug. The word “drug” as used herein is intended to encompass, for example, and without limitation, any medication, pharmaceutical, therapeutic, vaccine, aesthetic or other material which can be administered by injection. Actuation system 24 prepares the device for delivery of an injection and actuates delivery of an injection.

Delivery system 22 includes a drive assembly 26 to provide a driving force to effect an injection. In some versions of the device, a transmission assembly 28 may be provided to couple the nozzle assembly and the drive assembly.

Actuation system 24 includes a preparation assembly 30, such as a winder, to selectively arrange the drive assembly to provide a drive force to deliver an injection. A trigger assembly 32 assists a user in selectively actuating the drive assembly, directly or indirectly via the transmission assembly, to deliver an injection.

The structure and operation of needle-free injection devices configured to receive nozzle assembly 100 may include those disclosed in U.S. Published Patent Application No. 2005/0119608 and related U.S. patent application entitled “NEEDLE-FREE INJECTION DEVICE WITH AUTO-DISABLE,” filed Nov. 26, 2007. In the illustrative device shown in FIG. 1, drive assembly 26 includes a drive source 40, such as a spring, disposed between spring stop members 42, 44 such that bringing the spring stop members closer together compresses the spring, while decompression of the spring pushes the stop members away from one another. Relative rotation between housing sections, such as rotation of housing 16 relative to housing 14, actuates winder 30, which urges the distal spring stop towards the proximal spring stop to compress the spring. When the spring is compressed, the device is referred to as being in a wound configuration. In the example of FIG. 1, winder 30 may be rotated in a first direction and act on an internal winding nut 46 to translate a screw 48 relative to the winding nut, thereby moving the distal spring stop to the left.

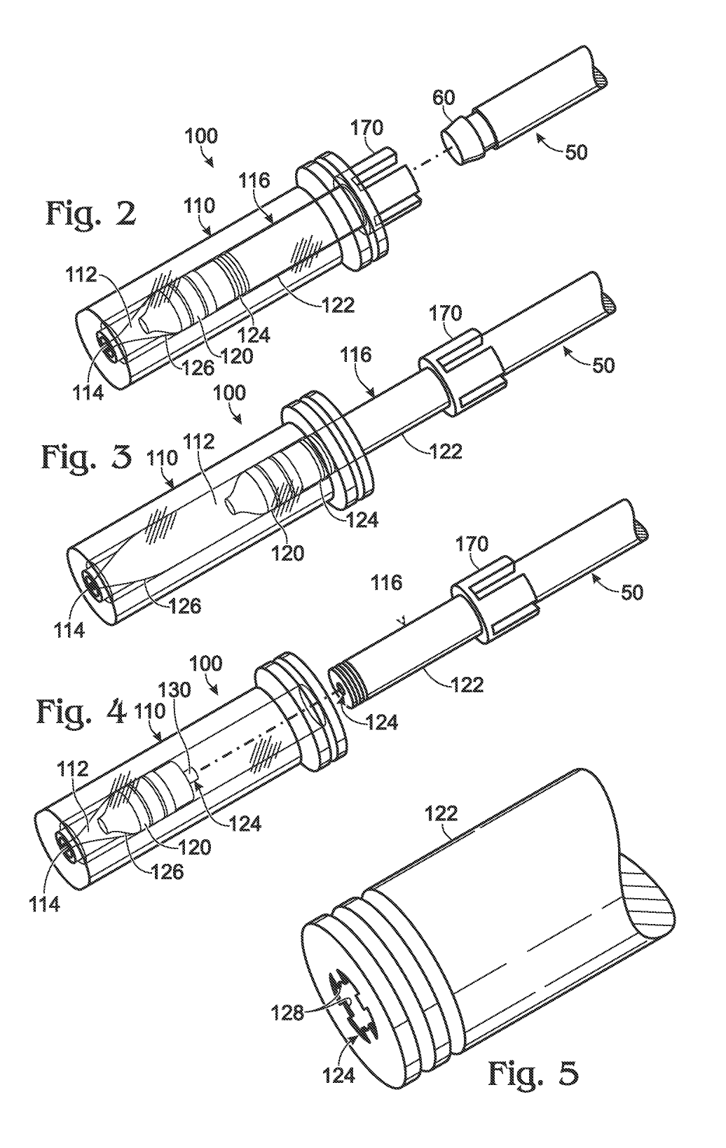

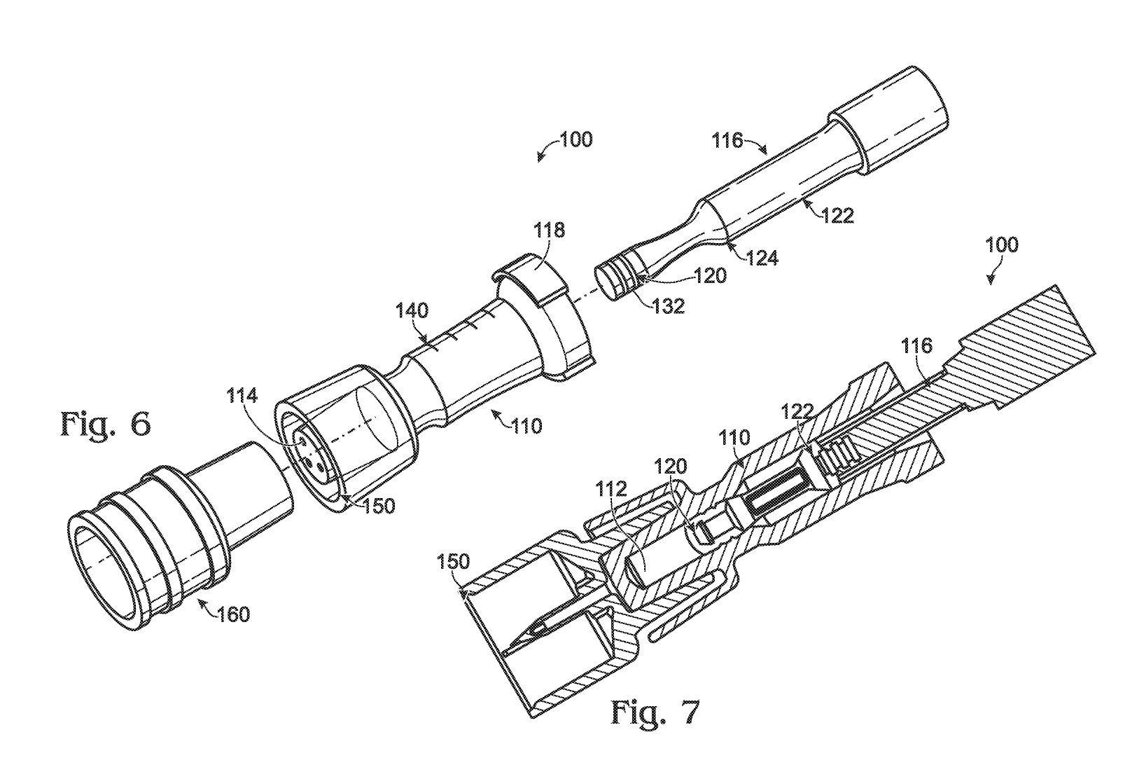

As also shown in FIG. 1, nozzle assembly 100 may be coupled to the device by placing the nozzle assembly through opening 20 in the device, such as by inserting the nozzle assembly along axis 18. The nozzle body may include one or more guides 118, as shown in FIGS. 2-4 and 6, to assist a user in locating the nozzle assembly relative to the device. The guide and opening may be similarly shaped to assist a user in aligning the nozzle assembly. For example, as shown in FIG. 6 the nozzle body may be configured to be inserted into the device and then rotated to lock the guides into the device.

In the example shown in FIG. 1, insertion of the nozzle assembly alters the configuration of the device so that an injection may be performed. Consequently, the device is disabled (i.e., prevented from releasing the spring) until a nozzle assembly is engaged. For example, the nozzle assembly of FIG. 1 moves the transmission assembly 28, such as in the form of a ram 50 that extends along the central axis of the device, to the right which allows one or more locking members 52 to engage the ram, thereby coupling the actuation system to the delivery system. Since rearward movement of the ram engages the proximal spring stop, the spring stop members are then coupled to one another and ready to be retracted relative to housing 14 to withdraw the ram and plunger, thereby drawing a dose into the nozzle body.

The rear housing 16 may be rotated in a second direction (opposite the first direction during spring compression) to withdraw the plunger and both spring stop members (to the right with respect to FIG. 1). Movement of the plunger to the right, as shown in FIG. 1, draws an injectate into chamber 112 through orifice(s) 114. During dosing, housings 14 and 16 may translate relative to one another as needed.

To deliver an injection, the trigger assembly 32, such as in the form of a button, is actuated to urge the ram and plunger towards the outlet orifice(s). For example, as the trigger assembly in FIG. 1 is pressed, a bushing 54 is urged towards the outlet orifices and provides a recess to receive locking members 52. The ram is therefore free to travel through the device. Since the distal spring stop is still held in place, decompression of the spring urges the proximal spring stop member towards the outlet orifice(s), which moves the ram and plunger towards the orifice(s) to deliver an injection.

In the example shown in FIGS. 1-4, nozzle plunger 116 includes first and second portions 120, 122 coupled together by a frangible region 124. The first portion 120 may be referred to as the proximal portion since it is closest to the outlet orifice. The second portion 122 may be referred to as the distal portion or base since it is further from the outlet orifice. The proximal portion may be configured to uncouple from the distal portion along the frangible region and lodge in a proximal end of the injectate chamber, thereby preventing intake of an injectate into the nozzle body. For example, to restrict reuse of the nozzle assembly, the proximal portion may remain in the injectate chamber, such as in a lead-in section 126 adjacent the orifice, upon retraction of the distal portion of the plunger from the injectate chamber.

The frangible region may be configured to yield in response to a force applied along a longitudinal axis of the plunger (along central axis 18, as shown in FIG. 1). For example, ram 50 may include an impact region 60 to apply a suitable force to the frangible region upon triggering of an injection. As shown in FIG. 1, as ram 50 moves toward outlet orifice(s) 114 and completes delivery of an injection. The continued force of impact region 60 against the plunger may urge distal portion 122 of the plunger forwards. However, since proximal portion 120 is prevented from moving further by the interior of the nozzle body, such as lead-in section 126, the frangible region breaks, as illustrated in FIG. 4. The proximal portion may become lodged in the nozzle body to prevent reuse of the nozzle assembly. Further, since there is no contact between the injectate and the distal portion, the distal portion may be removed from the ram without requiring a user to have contact with the injectate.

FIGS. 4 and 5 illustrate an example of a frangible region 124 after the proximal portion has been separated from the distal portion of the plunger. As shown, the frangible region includes fingers 128 that may be broken away, such as from a post 130, to separate the plunger portions.

As shown in FIGS. 1-4 and 6, the plunger may be at least partially visible through the nozzle body. The plunger may include first and second visibly distinct regions such that movement of the plunger through the nozzle body is measurable. For example, proximal portion 120 may include an over-molded tip 132, as best seen in FIG. 1, so that the tip is visibly distinct from the rest of the proximal portion. In other configurations, the proximal portion may be visibly distinct from the distal portion. Injectate chamber 112 may include a dose scale 140, as shown in FIG. 6, to incrementally measure the volume of the injectate drawn into the chamber. In some versions of the device, the dose scale includes indicia and the first and second visibly distinct regions of the plunger are configured to align with the indicia. Additionally or alternatively, the dose scale may be a pre-molded dose scale having ribs to indicate each unit of measure.

FIG. 6 further illustrates a nozzle assembly 100 suitable for delivering intradermal injections. The intradermal nozzle assembly may include several outlet orifices 114. For example, the nozzle assembly may include three orifices arranged in a triangular configuration, four orifices arranged in a square configuration, and the like. The outlet orifices may be laser drilled to produce orifice diameters that are smaller than those provided on typical nozzle assemblies. For example, the outlet orifices may have diameters equal to or smaller than 0.003 inch. The outlet orifices may be formed using the methods described in U.S. patent application Ser. No. 11/765,245, the disclosure of which is incorporated herein by reference.

As shown in FIGS. 6 and 7, plunger 116 includes a proximal portion 120 and a distal portion 122 having different diameters. For example, the distal portion may have a diameter that is larger than the diameter of the proximal portion. The reduced diameter portion acts as a pressure multiplier and allows for greater dose accuracy, such as for intradermal doses between 50 and 150 μL. For example, decreasing the plunger diameter while maintaining the spring force increases the pressure used to deliver an injection without changing the travel length of the ram and plunger. A multiple orifice nozzle in combination with a reduced plunger diameter therefore provides an increased delivery pressure from the same device. For example, the device disclosed in FIG. 1 may be coupled with nozzle assemblies having distal plunger portions with diameters suitable for coupling with transmission assembly 28, yet having proximal plunger portions with diameters suitable for delivering injections at different tissue depths. The device and corresponding spring 40 and spring travel length may be used with nozzle assemblies having proximal plunger diameters suitable for delivering intradermal, subcutaneous, and intramuscular injections. The reduced plunger diameter may enable use of a greater range of materials from which plunger 116 may be formed. For example, the plunger may provide first and second visibly distinct regions, as previously described, by using different plunger materials so that movement of the plunger through the nozzle body is more easily measurable, thereby providing greater dose accuracy. The two diameter plunger may be formed of different materials so that each diameter is formed of a plastic resin of different colors. For example, the plunger may be formed in an injection molding machine as a single piece using the process of “overmolding” or “two-shot molding” so that a portion of the plunger is a different color than the rest of the plunger.

The nozzle assembly may include a tension ring 150 for maintaining skin tension of a recipient during an injection. A vial adapter 160 may engage the nozzle body to couple the nozzle assembly to a vial of injectate during dosing of the nozzle assembly. The vial adapter may be coupled to a multiple orifice nozzle using a luer taper engagement.

Another way of preventing nozzle assembly reuse is by providing a nozzle assembly having an auto-disable that prevents the plunger and ram from being coupled together after an injection is performed. For example, a portion of the plunger may be deformable to restrict coupling of the plunger with the ram after delivery of an injection. In the following examples, the nozzle assembly is coupled to the device so that the plunger couples to the drive assembly, such as by snapping onto the ram. The device may then be wound, armed, and dosed as previously described to prepare for an injection. Once the device has been actuated, the ram may deform a portion of the nozzle assembly, such as a portion of the plunger, to prevent reuse of the nozzle assembly. The ram may be formed from a hard and/or substantially rigid material, such as steel, whereas the plunger may be formed from a brittle, soft and/or substantially deformable material, such as plastic, particularly high impact polysterene or polycarbonate.

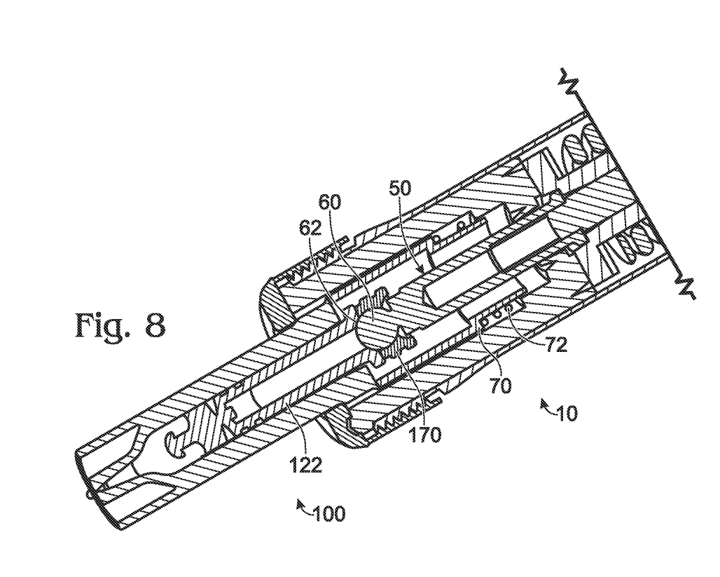

FIGS. 8-12 illustrate deformable plungers to restrict reuse of a nozzle assembly. Distal portion 120 of plunger 116 may include extensions 170 configured to couple the plunger to a drive assembly of a needle-free injection device. To restrict reuse of the nozzle assembly, the extensions may be configured to deform upon firing of the device, such as in response to a force applied along a longitudinal axis of the plunger. In the example shown in FIG. 8, ram 50 includes impact region 60 which is configured to apply a force to the plunger to deliver an injection and deform a set of extensions radially outward so that the plunger is unable to grip the ram. The ram is therefore unable to retract the plunger to draw a second dose into the nozzle assembly.

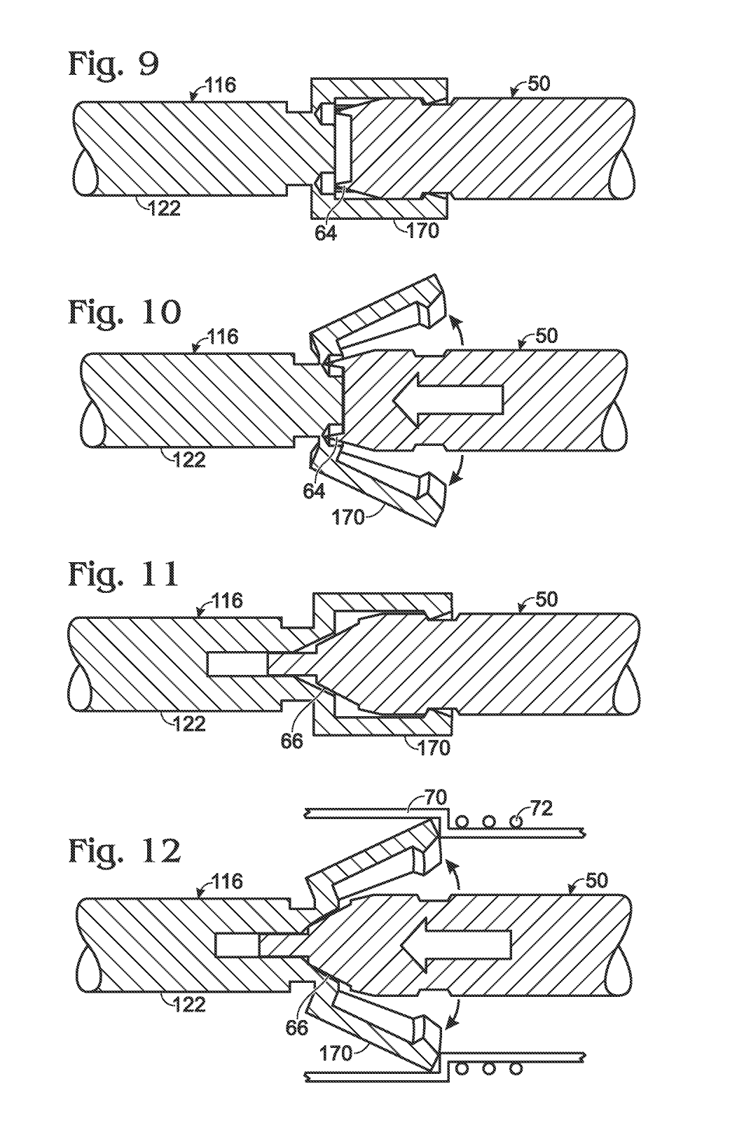

The extensions may be configured to couple the plunger to various geometries, such as to variously shaped ram impact regions 60. The impact region may include a curved portion 62 configured to urge the extensions away from the transmission member. For example, as shown in FIG. 8, the distal portion 122 of the plunger may include extensions configured to grip a spherical impact region of the ram that deforms the extensions outward to prevent the extensions from further gripping the ram. In the example shown in FIGS. 9 and 10, the impact region of the ram may include a sharp region, such as a cutting portion 64 that is configured to deform a set of extensions outward upon impact (as shown in FIG. 10). The ram may therefore deform a portion of the plunger through circumferential shear at the beginning of device actuation. In some configurations of the device, the ram may include an angled portion 66, such as a wedge-shaped impact region, that urges a set of extensions apart so that the ram is no longer gripped by the extensions once the device has been fired. The wedge may also be in the form of a separate member that is driven into the aft end (i.e., the distal portion) of the plunger to drive the extensions apart. This component may remain in the plunger to prevent the extensions from being forced back into place in an attempt to bypass the auto-disable mechanism.

As shown in FIGS. 1 and 8, the needle-free injection device may include a release mechanism 70, such as a ramp, to receive the deformed extensions. The ramp may be biased, such as by spring 72, to urge the plunger away from the ram, and thereby assist in removing the used nozzle assembly. For example, as illustrated in FIG. 12, once the extensions 170 are deformed outward, the extensions catch on ramp 70. Retraction of the ram would then merely pull the ram out of engagement with the plunger.

Although the present invention has been shown and described with reference to the foregoing operational principles and preferred embodiments, it will be apparent to those skilled in the art that various changes in form and detail can be made without departing from the spirit and scope of the invention. The present invention is intended to embrace all such alternatives, modifications and variances. The subject matter of the present invention includes all novel and non-obvious combinations and subcombinations of the various elements, features, functions and/or properties disclosed herein. Inventions embodied in various combinations and subcombinations of features, functions, elements, and/or properties may be claimed through presentation of claims in a subsequent application.

What is claimed is:

1. A nozzle assembly for a needle-free injection device comprising:
a nozzle body including an injectate chamber and one or more outlet orifices; and
a plunger configured to move through the injectate chamber toward the outlet orifice and including a distal portion and a proximal portion coupled together by a frangible region, wherein the proximal portion is configured to uncouple from the distal portion along the frangible region such that the proximal portion remains in the injectate chamber upon retraction of the distal portion from the injectate chamber.

2. The nozzle assembly of claim 1, wherein the proximal portion is configured to lodge in a proximal end of the injectate chamber, thereby preventing intake of an injectate into the injectate chamber.

3. The nozzle assembly of claim 1, wherein the frangible region is configured to yield in response to a force applied along a longitudinal axis of the plunger.

4. The nozzle assembly of claim 1, wherein the plunger is at least partially visible through the nozzle body and includes first and second visibly distinct regions such that movement of the plunger through the nozzle body is measurable.

5. The nozzle assembly of claim 4, wherein the nozzle body includes dose indicia and the first and second visibly distinct regions of the plunger are configured to align with the dose indicia.

6. A nozzle assembly for a needle-free injection device comprising:
a nozzle body including an injectate chamber and one or more outlet orifices; and
a plunger configured to urge injectate through the one or more outlet orifices and including extensions configured to couple the plunger to a drive assembly of a needle-free injection device, wherein the extensions are configured to deform in response to a force applied along a longitudinal axis of the plunger.

7. A needle-free injection device configured to receive the nozzle assembly of claim 6, wherein the device includes a drive assembly having a transmission member configured to couple with and urge the plunger through the injectate chamber to expel injectate through the one or more outlet orifices, the transmission member including an impact region configured to apply a force to the plunger and thereby deform the extensions.

8. The needle-free injection device of claim 7, wherein the impact region includes a curved portion configured to urge the extensions away from the transmission member.

9. The needle-free injection device of claim 7, wherein the impact region includes a cutting portion configured to urge the extensions away from the transmission member.

10. The needle-free injection device of claim 7, wherein the impact region includes an angled portion configured to urge the extensions away from the transmission member.

11. The needle-free injection device of claim 7, wherein the device includes a ramp configured to receive the deformed extensions and biased to urge the plunger away from the transmission member.

12. A nozzle assembly for a needle-free injection device comprising:
a nozzle body including an injectate chamber and one or more outlet orifices; and
a plunger configured to move through the injectate chamber toward the one or more outlet orifices and including a first portion and a second portion removably joined by a frangible region, the plunger further including extensions configured to couple the plunger to a drive assembly of a needle-free injection device.

13. The nozzle assembly of claim 12, wherein the first and second portions are configured to uncouple along the frangible region and at least one of the first and second portions is configured to remain in the injectate chamber upon completion of an injection.

14. The nozzle assembly of claim 12, wherein the frangible region is configured to yield in response to a force applied along a longitudinal axis of the plunger.

15. The nozzle assembly of claim 12, wherein the extensions are configured to deform in response to a force applied to the plunger.

16. A needle-free injection device configured to receive the nozzle assembly of claim 12, wherein the device includes a drive assembly having a transmission member configured to couple with and urge the plunger through the injectate chamber to expel injectate through the one or more outlet orifices, the transmission member including an impact region configured to apply a force to the plunger and thereby deform the extensions.

17. The needle-free injection device of claim 16, wherein the impact region includes a curved portion configured to urge the extensions away from the transmission member.

18. The needle-free injection device of claim 16, wherein the impact region includes a cutting portion configured to urge the extensions away from the transmission member.

19. The needle-free injection device of claim 16, wherein the impact region includes an angled portion configured to urge the extensions away from the transmission member.

20. The needle-free injection device of claim 16, wherein the device includes a ramp configured to receive the deformed extensions and biased to urge the plunger away from the transmission member.

21. A nozzle assembly for a needle-free injection device comprising:
a nozzle body including an injectate chamber and one or more outlet orifices; and
a plunger configured to move through the injectate chamber toward the outlet orifice and including a distal portion having a first diameter and configured to engage a needle-free injection device and a proximal portion having a second diameter and configured to urge an injectate through the one or more outlet orifices, wherein the first diameter is greater than the second diameter.

22. The nozzle assembly of claim 21, wherein the plunger is at least partially visible through the nozzle body and includes first and second visibly distinct regions such that movement of the plunger through the nozzle body is measurable.

23. The nozzle assembly of claim 22, wherein the nozzle body includes dose indicia and the first and second visibly distinct regions of the plunger are configured to align with the dose indicia.

24. The nozzle assembly of claim 21, wherein the one or more outlet orifices each have a diameter not more than approximately 0.003 inch.
